(19) United States
(12) Patent Application Publication
(10) Pub. No.: US 2012/0298047 A1
(43) Pub. Date: Nov. 29, 2012

(54) TANNING DEVICE AND METHOD

(75) Inventor: Nicola Gheda, Brescia (IT)

(73) Assignee: BRONZE ITALIA S.R.L., 80144 Napoli (IT)

(21) Appl. No.: 13/574,765

(22) PCT Filed: Oct. 7, 2010
(86) PCT No.: PCT/IB10/54537

§ 371 Date: Aug. 16, 2012

(30) Foreign Application Priority Data
Jan. 25, 2010 (IT) BS2010A000008

(51) Int. Cl. A61M 35/00; B65D 83/14

(52) U.S. Cl. 119/604

(57) ABSTRACT

A tanning device (1) and related method, the device comprising a resting base (2) for a user and a supporting frame (3) associated with said base (2); the frame (3) is configured for housing a plurality of delivery nozzles of a spray delivery system of a tanning substance, where said base (2) comprises a support (12) with substantially flat shape for enabling the user to lie down for receiving the tanning substance delivered from said delivery nozzles.

The present invention relates to a tanning device and a related method, particularly through the use of tanning substances,

The present invention finds application in devices for artificial tanning, i.e. apt to cause an artificial (i.e. without sun exposure) skin color alteration, typically used in the field of beauty treatments or aesthetic body care as are usually performed in commercial premises such as tanning centers, aesthetic centers, beauty centers.

For instance, there are tanning devices with UV-ray emission to the user who undergoes the tanning treatment, which stimulates melanogenesis, i.e. melanin production by melanocytes.

Conversely, a different type of devices make use of suitable chemical substances, typically known as “tanning substances” or “self-tanning products”, which are applied (typically sprayed) onto the users skin. The tanning substance used in these devices is typically contained in a mixture of other substances, such as for instance water, glycols, preserving agents or moisturizing substances, and represents the active substance apt to cause a skin color alteration. Among the tanning substances on the market, which are also available in spray containers provided with a dispensing cap for personal and domestic use, dihydroxyacetone (DHA) can be mentioned by way of example, which is a carbohydrate obtained e.g. as sugar cane extract and widely used today in the field of spray tanning.

Tanning substances penetrate surface skin layers making skin darker and the darkening effect progressively ceases after some days. They do not modify the structure of skin cells and do not cause melanin release, but only act on the surface by forming a layer with a darker color than the natural color of the user's skin. Tanning substances, therefore, allow to eliminate the problems related to the use of UV rays, such as e.g. the risk of appearance of melanomas and skin tumors and the risk of burns due to an excessive exposure to such rays.

The devices making use of these tanning substances, typically referred to as “spray tanning devices”, can be actuated by an operator or be of automatic type. In the first case, the tanning substance which is able to darken skin is contained in a tank and sprayed onto the user's skin by means of a manual dispenser, typically a spray gun similar to those used for spraying paints of various type, which is manually pointed by the operator around the user's body so as to distribute the tanning substance. The dispenser is connected, typically by means of a flexible tube, to a source of compressed air (e.g. a compressor), which is able to atomize the tanning substance contained in the tank and let it get out from a dispenser nozzle. This operation is usually performed inside a suitable tent or cab in which the user is standing and which is provided with an opening through which the operator points the spray towards the user's skin from outside such tent or cab; in order to cover with the tanning substance the whole body surface to be darkened, the user can move for facing the operator with parts of his/her body that have not yet been reached by the atomized tanning substance.

It is further known about spray tanning devices of automatic type, typically for integral tanning, made up of a cab which is similar in structure to a tanning cab with UV rays. These devices are commonly referred to as “tanning showers” and are made up of a closed or half-closed cab inside which the user is standing. In the case of spray tanning, the side walls of the cab are equipped with a certain number of nozzles for the emission of the tanning substance, uniformly arranged so as to point atomized sprays that can cover the whole exposed body surface of the user who has to undergo the tanning treatment.

Also these automatic devices are equipped with a compressor apt to supply the required pressure, and with ducts conveying to the nozzles the tanning substance from one tank, but they do not require the presence of an operator since the plurality of nozzles in the cab enables to reach the various parts of the user's body. The Applicant has found that current spray tanning devices and related tanning methods are not without drawbacks and can be improved under various points of view, mainly concerning the possibility of performing a uniform delivery and of maintaining over time functionality and performance of said device.

As a matter of fact, it should be pointed out that in the aforesaid devices provided with tank and compressor, during and after the delivery of the tanning substance, solid deposits and sediments are formed in the ducts and above all in the dispensing nozzles, which deposits and sediments cause a partial or total obstruction of the ducts and/or nozzles. In order to overcome this drawback, complex maintenance operations are usually carried out on traditional spray tanning devices, which necessarily occur by disassembling the parts which the tanning substance gets through (e.g. nozzles, ducts, fittings) and by cleaning them, then the device must be re-assembled for subsequent use. This involves high costs related to the use of known devices, due to the long downtime of the device required for continuous maintenance operations and to the use of skilled maintenance personnel.

The Applicant has found that current spray tanning devices or methods have further drawbacks, particularly concerning the possibility of reaching the whole user's skin portion to be darkened with the tanning substance.

Another drawback of known devices, in particular of cab-shaped spray tanning devices, concerns the large amount of tanning substance which is wasted during use, i.e. which does not reach the user's skin. This is particularly relevant for cab-shaped devices in which the user is standing and nozzles atomize the tanning substance sideways with respect to the user, wherein a considerable part of atomized tanning substance flows downward while progressively getting away from the nozzle, due to the progressive loss of speed and to the simultaneous effect of gravity, and deposits onto the cab bottom before reaching the user's body.

It should be further pointed out that known methods involve atomizing the tanning substance onto the user's skin while the latter is standing, which may not enable to uniformly reach those skin portions that are hidden or covered because of skin folds formed by gravity. This is even more evident for users with a large body mass and/or for aged users. Moreover, this phenomenon is particularly evident on the abdomen wherein skin and/or breast folds and recesses are more concentrated.

Under these circumstances, the technical task underlying the present invention consists in providing a tanning device and a related method which can overcome one or more of the drawbacks mentioned above. One of the possible aims of the present invention in one or more of its various aspects consists in providing a tanning device and a related method which can uniformly deliver the tanning substance and maintain function and performance over time, reducing the need for complex and/or frequent maintenance and/or cleaning operations, in particular the need to disassemble, clean and re-assemble the hydraulic circuit for tanning substance delivery.

A further possible aim of the present invention in one or more of its various aspects consists in providing a tanning device and a related method which can reach and cover with the tanning substance in a uniform manner the whole area of the user's skin to be darkened.

A further possible aim of the present invention in one or more of its various aspects consists in providing a tanning device and a related method which can point and direct at best, i.e. towards the user's body, the tanning substance, avoiding waste as much as possible.

A further possible aim of the present invention in one or more of its various aspects consists in providing a tanning device and a related method which are in use more convenient and comfortable for the user and/or operator.

Another possible aim of the present invention in one or more of its various aspects consists in providing a tanning device which is simple and/or cheap in design and/or construction and/or installation and/or configuration and/or operation and/or maintenance.

One or more of the above aims and others which will be more apparent in the following description are basically achieved by a tanning device and a related method, comprising the technical features contained in one or more of the appended claims, each of them being considered alone (without its dependent claims) or in any combination with other claims, and in the following aspects, variously combined, also together with the aforesaid claims.

In one aspect the invention relates to a tanning device comprising a resting base for a user and a supporting frame associated with said base and configured for housing a plurality of delivery nozzles of a spray delivery system of a tanning substance, where said base comprises a support with substantially flat shape for enabling the user to lie down for receiving the tanning substance delivered from said delivery nozzles. In one aspect, said spray delivery system comprises a tank containing said tanning substance and a source of pressure for spraying said tanning substance from said delivery nozzles toward the user in a direction from the top toward the bottom, said frame comprising a framework overlying said support and provided with said plurality of nozzles, so that the tanning substance is delivered towards said support, the framework being able to translate with respect to said support in a direction parallel to a direction of longitudinal development of said support.

In one aspect, the frame comprises a plurality of seats apt to removably house a plurality of spray containers for said tanning substance and provided with said delivery nozzles for delivering said tanning substance automatically towards the user, said frame comprising a framework overlying said support and provided with said plurality of seats, so that the tanning substance is delivered towards said support, the framework being able to translate with respect to said support in a direction parallel to a direction of longitudinal development of said support.

It is therefore possible to replace the spray container, thus overcoming maintenance difficulties of the prior art.

In one aspect, said frame is configured for removably housing a plurality of spray containers for a tanning substance and for delivering said tanning substance from said plurality of spray containers towards the user. It is thus possible to obtain a simultaneous and uniform delivery onto a larger surface of the user's body and to speed up the tanning treatment.

In one aspect, said frame comprises at least one suitably shaped seat apt to removably house said at least one spray container or a plurality of seats for said plurality of spray containers. It is thus possible to easily associate or remove said at least one spray container or said plurality of spray containers with or from said frame.

In one aspect, said base comprises a support with a basically flat shape and horizontal development for enabling the user to lie down along a direction of longitudinal development of said support for receiving the tanning substance delivered from said at least one spray container or said plurality of spray containers. It is thus possible to avoid lighter skin areas which are formed with the user standing because of close tissues overlapping.

In an alternative aspect, the aforesaid support consists of a back of a chair structured for allowing the user to be in a lying position for receiving the tanning substance supplied by said plurality of spray containers or nozzles. It is thus possible to deliver the tanning substance on the face and/or on the upper part of the user body.

In one aspect, said frame comprises a framework overlying said support and equipped with said at least one seat or plurality of seats, so that the tanning substance is delivered towards said support. It is thus possible to accurately direct the tanning substance onto the user's body avoiding waste of tanning substance.

In one aspect, said frame comprises one or more supporting uprights for said framework, which project from the base.

In one aspect, said framework comprises a sliding rail developing along said longitudinal development of the support, a trolley equipped with said at least one seat or plurality of seats and slidingly engaged with said rail, and moving means for said trolley along said rail. Thus, by enabling a relative movement between spray containers and user it is possible to reach with one spray container only or with a limited set of spray containers the whole body surface of the user.

In one aspect, said framework comprises a cover developing above said base, said cover being able to be switched between a closed configuration in which it defines an internal volume for housing the user between an internal surface of said cover and said support, and an open configuration in which it enables the user's arrangement on said support and the disengagement thereof from said device. It is thus possible to perform a tanning treatment inside a basically closed or limited space and avoid waste of tanning substance and/or unwanted contamination due to the mixture containing the tanning substance.

In one aspect, said frame further comprises actuating means configured for acting upon a respective dispensing element of said at least one spray container or of said plurality of spray containers, for automatically delivering the tanning substance from said at least one spray container. It is thus possible to provide a tanning device which is able to operate automatically.

In one aspect, said actuating means comprise at least one control element shifting between at least a first position in which it is not apt to interact with said respective dispensing element, and a second position in which it is apt to push said dispensing element so as to obtain said automatic delivery.

In one aspect, said actuating means comprise a slide having at least one seat, which can shift between at least a first position in which said dispensing element is not apt to interact with a respective counter-element and a second position in which said dispensing element is pushed against a respective counter-element.

In one aspect, the tanning device comprises said at least one spray container or said plurality of spray containers, each container containing a pressurized mixture containing at least said tanning substance, typically dihydroxyacetone (DHA), and being equipped with a dispensing element for atomizing said mixture.

In one aspect, the tanning device further comprises a source of an electromagnetic radiation. It is thus possible to add to the tanning effects of the tanning substance the tanning effects resulting from the exposure to an electromagnetic radiation, typically UV radiation.

In one aspect, the tanning device further comprises a heat source configured to heat the skin of the user.

Preferably, the heat source is a generator of a stream of hot air, having a temperature preferably greater than 30° C., more preferably greater than 35° C., or a source of an infrared radiation. This can enable to reduce the drying time of the mixture supplied from the spray container or from the plurality of spray containers onto the user's skin, thus improving the penetration of the tanning substance into the skin layers and achieving a more effective tanning treatment.

In one aspect the tanning device further comprises a suction system, comprising for example a suction fan and a filter, configured for aspiring a quantity of tanning substance delivered from said at least one tanning spray container or said plurality of spray container, for example during or at the end of a tanning treatment. This can avoid the substance to move away from the device and disperse into the surrounding environment, thereby soiling the environment itself.

In one aspect the tanning device is made so as to allow also the implementation of not-tanning cosmetics treatments (e.g. massage, hair removal, etc.) on a user positioned on said support. It is thus possible to use the tanning device for a variety of aesthetics treatments allowing to reduce the space, for example in a aesthetic center, necessary to accommodate different devices used for the different aesthetics treatments.

In another aspect, the invention relates to a tanning method comprising the following steps:
arranging the aforesaid tanning device in any one of the aspects of the present invention;
placing said at least one spray container in said at least one seat (or plurality of seats);
delivering the tanning substance towards at least one area of the user's body from a respective dispensing element of said at least one spray container (or of said plurality of spray containers).

In one aspect, the method further comprises the steps of removing, typically upon exhaustion of the tanning substance contained in said at least one spray container or in one or more spray containers of said plurality of spray containers (or upon obstruction of the corresponding dispensing element), said at least one spray container or said one or more spray containers of said plurality of spray containers from said frame, and associating with said frame a new spray container or several new spray containers containing tanning substance to be supplied. It is thus no longer necessary to arrange and maintain a hydraulic system for supplying the tanning substance and there is less need for complex and/or frequent maintenance and/or cleaning operations.

In one aspect, the method comprises the step, to be performed by the user, of switching from a lying supine position to a lying prone position, so that the tanning substance can reach and cover the whole body surface of the user.

In one aspect, during said step of delivering the tanning substance the latter is delivered downwards.

In one aspect, the method further comprises the step of actuating through said actuating means said dispensing element of said at least one spray container or said respective dispensing elements of said plurality of spray containers, so as to automatically deliver the tanning substance from said at least one spray container or said plurality of spray containers.

Further characteristics and advantages will be more apparent from the detailed description of some embodiments, among which also a preferred embodiment, by way of non-exclusive example of a tanning device and related method according to the present invention. This description will be made in the following with reference to the accompanying drawings provided to a merely indicative and therefore non-limiting purpose, in which:

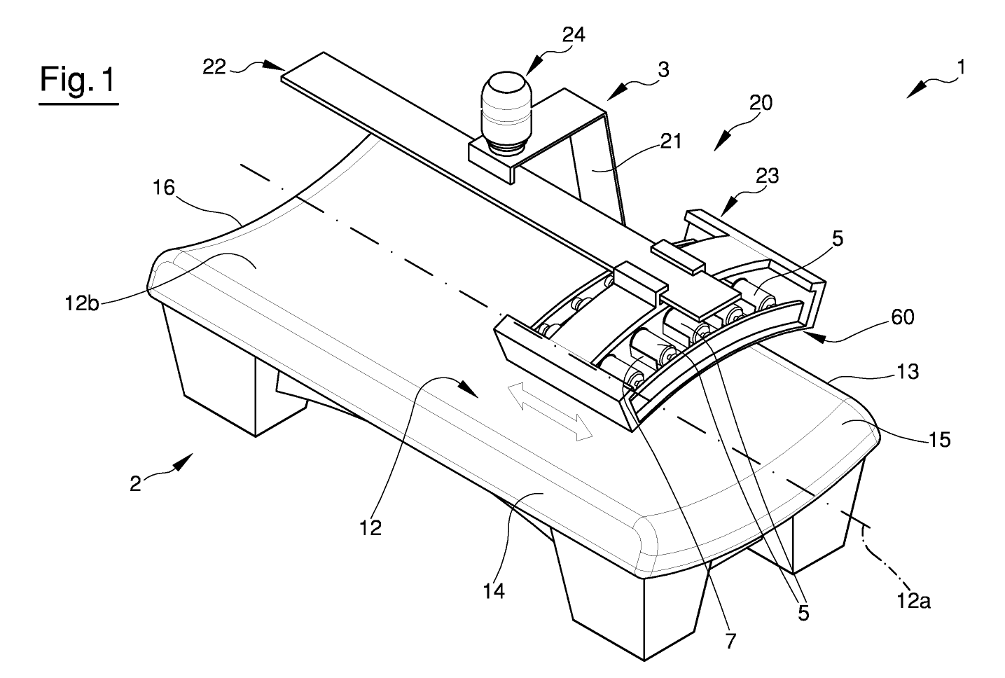

FIG. 1 is a perspective view of a tanning device according to the present invention;

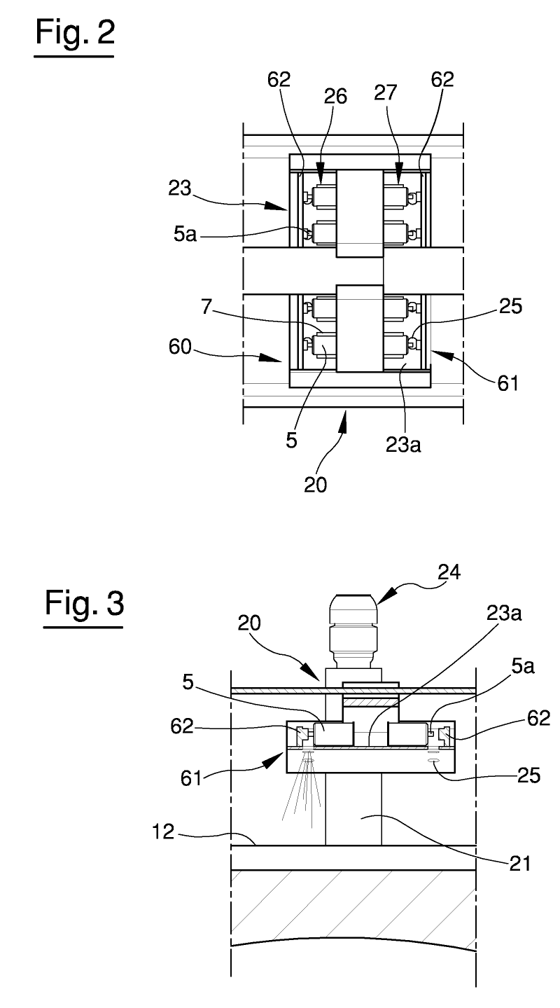

FIG. 2 is a partial top view from above of the tanning device of FIG. 1;

FIG. 3 is a partial view in lateral elevation and sectioned of the tanning device of FIG. 1;

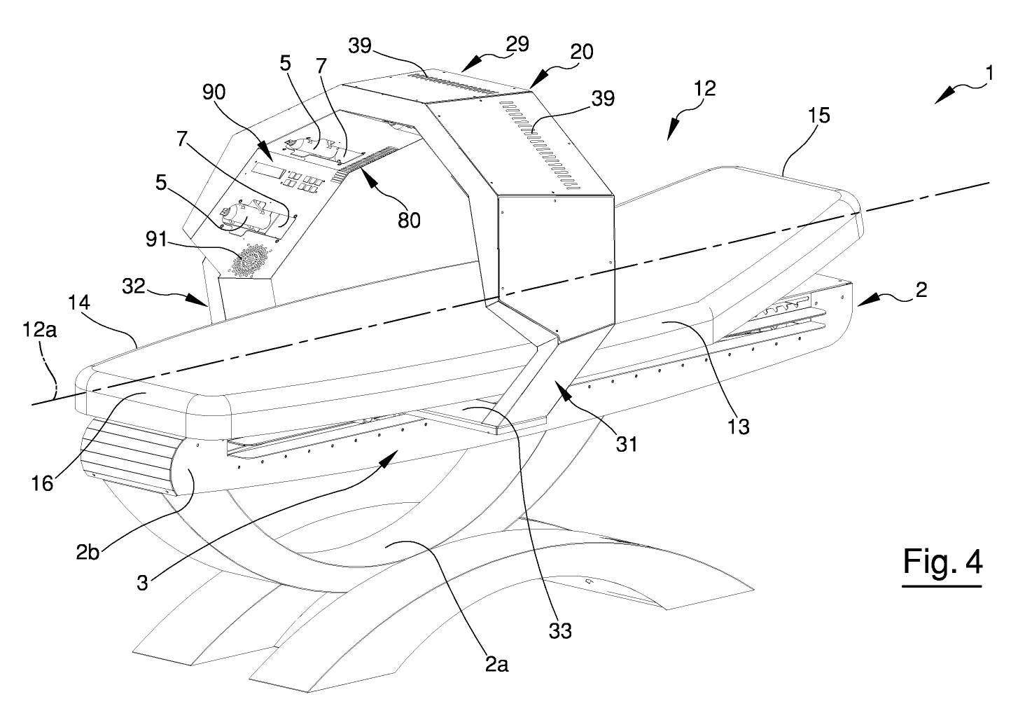

FIG. 4 is a perspective view of another possible embodiment of a tanning device according to the present invention;

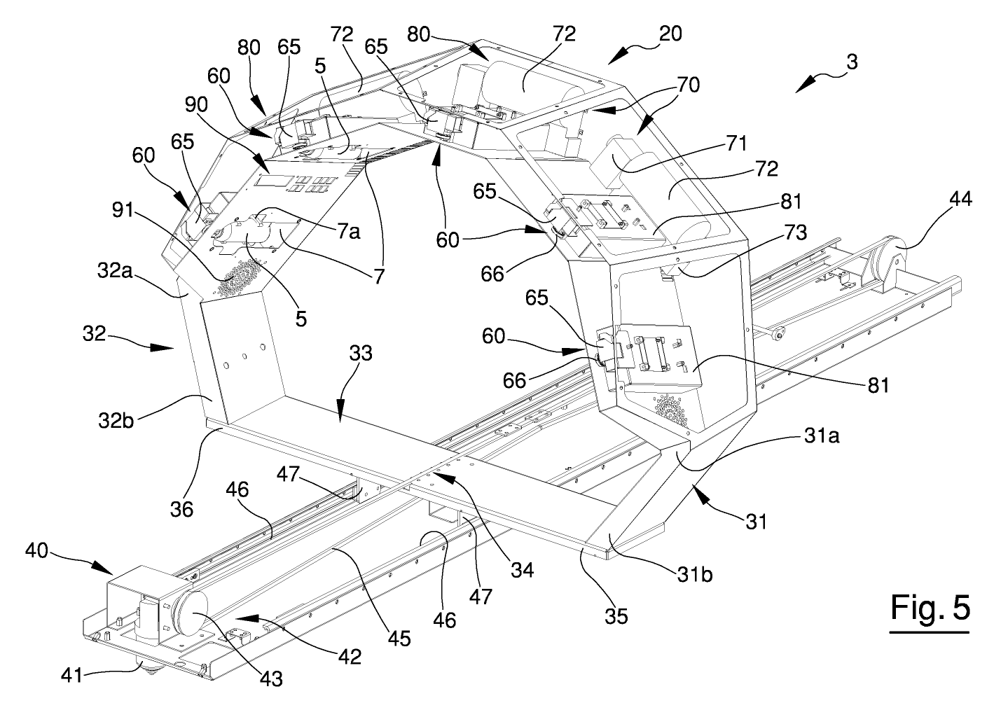

FIG. 5 is an another perspective view of the device of FIG. 4, with some parts removed;

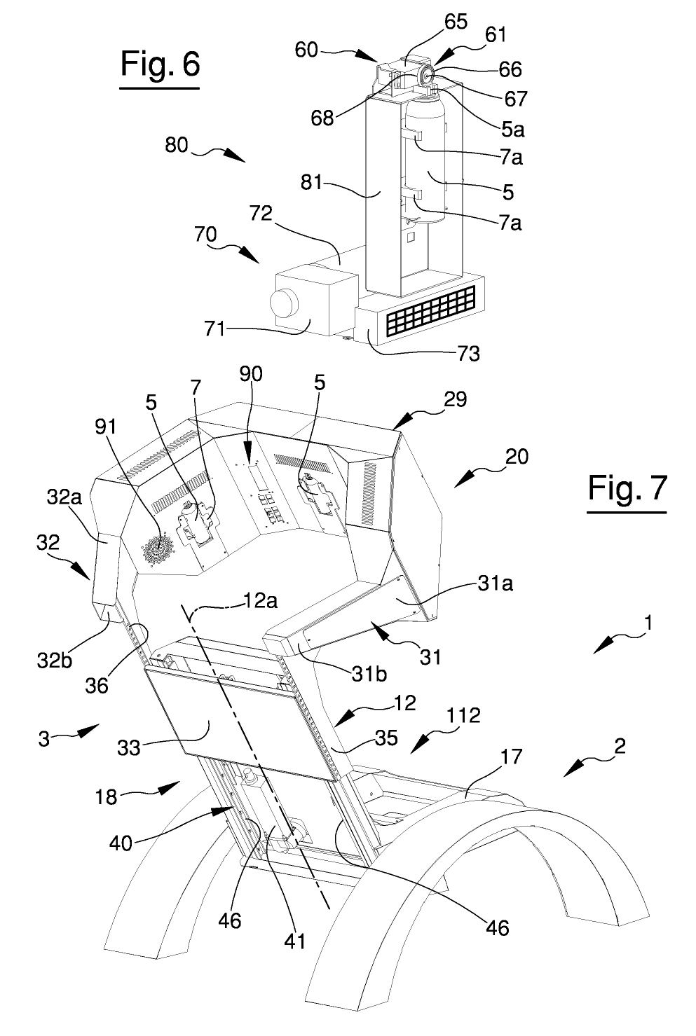

FIG. 6 is a perspective view showing a part of the device of the present invention;

FIG. 7 is a perspective view of a still another possible embodiment of a tanning device according to the present invention, with some parts removed.

With reference to the accompanying figures, a tanning device according to the present invention is globally referred to with numeral 1. In general, the same numeral is used for identical or similar elements, if required in their variants of embodiment.

The device 1 comprises a resting base 2 for a user and a supporting frame 3 associated with the base. This frame 3 is configured for removably housing at least one spray container 5 for a tanning substance and for delivering this tanning substance from the spray container 5 towards the user. Preferably, as shown by way of example in the figures, the frame 3 is configured for removably housing a plurality of spray containers 5 for tanning substance and for delivering the tanning substance from this plurality of spray containers 5 towards the user.

Typically, these spray containers are made up of bombs enclosing inside a pressurized mixture which is atomized outside by means of a suitable dispensing element 5a. Examples of pressurized spray containers containing tanning substances (such as DHA) are known.

Preferably, the frame 3 comprises at least one seat 7 of suitable shape apt to removably house the aforesaid spray container 5 or a plurality of seats 7 for the aforesaid plurality of spray containers 5. As shown by way of example in the figures, the seats 7 can preferably match the shape of the spray containers 5, so as to enclose them at least partially, typically on the bottom of said containers and part of the side surface, thus firmly locating them on the frame 3 and simultaneously enabling a simple manual removal thereof.

Preferably, as shown by way of example in FIGS. 1 and 4, the base 2 comprises a support 12 having a basically flat shape and horizontal development for enabling the user to lie down along a direction of longitudinal development 12a of such support, in order to receive the tanning substance supplied from the spray container 5 or from the plurality of spray containers 5.

Preferably, as shown by way of example in FIGS. 1 and 4, the support 12 is basically rectangular in shape and has a resting surface 12b, two longer sides 13 and 14 extending parallel to the direction of longitudinal development 12a and between which the user is lying, and a first 15 and a second shorter side 16 substantially perpendicular to the direction of longitudinal development 12a, on which the user's head and fee are located, respectively. Preferably, the support 12 is symmetrical with respect to the direction of longitudinal development 12a and the longer sides 13 and 14 are equivalent to one another. Analogously, the shorter sides 15 and 16 can be homologous. Preferably, in an embodiment not shown in the figure, the surface 12b of the support, on which the user lies down, can be ergonomically shaped so as to support the various body parts and provide a more comfortable rest for the user lying thereon, if required with massage functions. Preferably, the support 12 can be made of plastics, glass, metal or any other suitable material, or can be covered with a soft coating, a fabric or other material.

As shown by way of example in FIGS. 1, 2, 3, 4, 5 and 7, the frame 3 comprises a framework 20 lying above the support 12 and equipped with the aforesaid seat 7 or plurality of seats 7, so that the spray container 5 or the plurality of spray containers are located above the support 12. Thus, the tanning substance is advantageously delivered from top to bottom and is directed precisely towards each body part of the user lying down.

Preferably, as shown by way of example in FIGS. 1 and 3, the frame 3 comprises one or more supporting uprights 21 for the framework 20, which project from the base 2. As shown by way of example in the embodiment of FIG. 1, the frame comprises a single middle upright associated with the base on one of the two longer sides 13 or 14. In an alternative embodiment (not shown), the framework 20 can be supported by several uprights, e.g. two, projecting from the base on the same longer side 13 or 14.

Preferably, the uprights 21 can be connected to the base 2 by means of an articulated hinge (not shown) enabling a movement of the upright, and therefore of the framework, with respect to the base and allowing to lead these structures away from the surface 12b of the support 12, e.g. to enable to user to access the support 12 and to disengage from the latter in a comfortable and safe manner, avoiding possible collisions between the user and parts of the frame.

In an alternative embodiment not shown, the framework 20 is fastened to the ceiling above the device 1 by means e.g. of cables, thus avoiding the presence of posts.

Preferably the framework 20 comprises a sliding rail 22 developing along the longitudinal development 12a of the support 12, and a trolley 23 equipped with the aforesaid seat 7 or with the plurality of seats 7 and slidingly engaged with the rail 22. Advantageously, the framework 20 further comprises movement means 24 (schematically shown in the figure) for the trolley 23 along the rail 22. These movement means can advantageously comprise at least one motor placed on the framework 20 and apt to cause the trolley 23 to shift along the rail 22, typically in both directions of the longitudinal development 12a according to a to-and-fro motion, i.e. shifting between the shorter sides 15 and 16 of the support 12. Thus, the trolley 23, equipped with the seats 7 housing the spray containers 5 and lying above the support 12, can face during the treatment the surface 12b in its whole extension, so as to deliver the tanning substance on the whole longitudinal development of the user's body.

Preferably, the movement means 24 can further comprise a mechanical drive (not shown) of known type, which can transmit motion from the motor to the trolley 23. This drive can be for instance a rack-and-pinion coupling, wherein a pinion is integral with a drive shaft of the motor, the latter being mounted on the trolley 23, and engages a rack placed on the rail parallel to the direction of longitudinal development. In this case the rotation of the drive shaft causes the shift of the trolley engaged into the rail.

As an alternative, the motor can transmit motion to a pair of pulleys causing the movement of a belt, e.g. a toothed belt, with which the trolley is integral.

Preferably, the plurality of seats 7 is distributed on the framework 20 uniformly or according to a specific pattern, so as to cause a uniform delivery of the tanning substance by the plurality of spray containers. By way of example, if the framework 20 comprises the aforesaid trolley, as shown by way of example in FIG. 1 or 2, the plurality of seats 7 is arranged so as to house two opposed rows 26 and 27 of spray containers. These rows are preferably parallel to the aforesaid shorter sides 15 and 16 and enable to deliver the tanning substance basically from one longer side 13 to the other longer side 14. Moreover, the shift of the trolley 23 along the rail 22 by the aforesaid movement means 24 allows to cover with the delivery the whole length, along the direction of longitudinal development 12a, of the support 12 and thus the whole surface 12a.

Each row of seats can house for instance spray containers containing the same mixture of tanning substance and apt to cause on the user's skin a particular degree of color alteration and therefore of tanning. By way of example, the two rows 26 and 27, arranged on opposed sides of the trolley 23, produce two different degrees of artificial tanning, e.g. light and intense tanning, respectively.

In an alternative embodiment, not shown, the framework comprises a cover developing above the base 2. Preferably, this cover can switch at least between a closed configuration, in which it defines an internal volume for housing the user between an internal surface of the cover and the surface 12a of the support 12, and an open configuration in which it enables the user's arrangement on the support 12 and the disengagement thereof from the device. Preferably, the cover can be associated with the base 2 on one of the longer sides 13 or 14 of the support 12 by means of a hinge (not shown) which allows the cover to switch between the aforesaid closed and open configurations.

Preferably, the plurality of seats 7 is distributed in a fixed manner on the internal surface of the cover uniformly or according to a specific pattern, e.g. in points lying at the same distance and facing the surface 12a of the support 12, so that the delivery of the tanning substance by the plurality of spray containers covers the whole surface 12a of the support 12.

In an alternative embodiment, the aforesaid framework 20 equipped with rail 22 and mobile trolley 23 can be fastened to the internal surface of the aforesaid cover.

In a possible further alternative embodiment the uprights can project from the basis on opposed sides with respect to the direction of longitudinal development 12a, i.e. from the longer sides 13 and 14, for supporting the framework 20.

For instance, in an embodiment exemplary shown in FIGS. 4 and 5, the support has a substantially horizontal development and the aforesaid trolley consists of a portal structure 29 comprising the aforesaid framework, a support member and two uprights 31 and 32 in support of the framework 20 arranged on opposite sides with respect to the support, for example projecting from the two longer sides 13 and 14 in two corresponding facing positions. Preferably the support member lying below the support and preferably it is provided with two end portions 35 and 36 opposed each other and stiffly fixed each to a respective lower end of the respective upright. Preferably, this portal structure develops transversally to the direction of longitudinal development 12a and the framework extends at a height above the support 12 apt at least to enable the user to lie down on such support. Thus the overall structure of the device results advantageously rational, with a small weight and encumbrance.

Preferably, the movement means 40 are structured for moving the support member 33 with respect to the base and for determining the translation of the portal structure 29 parallel to the direction of longitudinal development 12a

Preferably the frame comprises two guides 46, developing parallel to the longitudinal development in correspondence of the longitudinal opposed sides of the support, and the support member 33 comprises at its two end portions 35 and 36 two sliders 47 slidingly inserted into the respective guide. Thus the translational movement of the portal structure is parallel to said longitudinal development, stable and aligned. In addition, the coupling guide-slider allows to support the load of the portal structure which weigh down on the frame. The presence of two guides 46 advantageously allows to obtain a support of the portal structure into two distinct points, which is stable and safe and realizes a symmetrical distribution of loads on the frame. In addition, the placement of the two guides 46 below the support advantageously allows to obtain a safe movement of the portal structure, because the uprights and the framework are sufficiently spaced (on side and on top respectively) from the user, and the support member slides below the support and does not interfere with the surface of the latter. This will prevent the user of the device to get in contact, during the treatment, with moving parts of the portal structure.

In an alternative embodiment not shown, the support element 33 consists of the aforesaid two end portions 35 and 36, each equipped with the respective slider, distinct and separate and each fastened to the respective upright.

Preferably, the shifting means can comprise a motor 41 acting directly, or by means of a mechanical drive, upon the support member 33 so as to cause the shift thereof parallel to the direction of longitudinal development 12a. Preferably the guides 46 are arranged in correspondence with one or both longer sides 13 and 14 between the first shorter side 15 and the second shorter side 16.

The portal structure allows to cover with continuity and precision the entire surface of the support, delivering the tanning substance toward successive portions of the support itself, maintaining however a limited encumbrance along said direction of longitudinal development. The portal structure can be advantageously positioned, for example when the treatment is completed, in correspondence of one of the two longitudinal ends of the support (typically one of the aforesaid shorter sides 15 or 16), leaving clear the volume above the support.

Preferably the two uprights 31 and 32 have a respective upper end 31a and 32a stiffly fixed to the framework 20 and a respective lower end 31b and 32b opposed to the respective upper end. Preferably the support member 33 is provided with a central mounting portion 34 interposed between the two end portions 35 and 36.

Preferably the movement means comprise a mechanical transmission 42, preferably positioned entirely below the support, which includes a driving pulley 43, mechanically coupled to the motor 41, a driven pulley 44, positioned on the opposite side of the framework with respect to the driving pulley, and a transmission member 45 (exemplary a belt or a chain) connecting the driving and driven pulleys, the mounting portion 34 of support member 33 being stiffly fixed (preferably at the bottom) to the transmission member 45 in such a way that the rotation of the motor determines the sliding of the sliders 47 in the guides 46 and the subsequent translation of the portal structure along a direction parallel to the longitudinal development of the support.

In an alternative aspect not shown, the movement means comprise a linear motor developing parallel to the direction of longitudinal development 12a and configured for determining the translation of the support member 33 of the portal structure. Preferably, the linear motor consists in the coupling of the guide 46 with the respective slider 47, wherein the guide and the slider are respectively the stator and the rotor of the linear motor. Preferably, the frame comprises two linear motors, one for each of the two guides 46.

Preferably, the base 2 includes a lower basement 2a and a box-shaped housing 2b fixed above the basement and structured for containing the movement means 40, the support being mounted above the box-shaped housing. Preferably, the driving pulley and the driven pulley are positioned in correspondence of opposed longitudinal ends of the box-shaped housing. Preferably, the guides are fixed to the box-shaped housing.

Preferably, the movement means comprise one or more sensors (not shown, exemplary an encoder, a resolver, a linear potentiometer or an optical scale) configured for detecting the position of the portal structure, preferably the relative position with respect to the support or the frame. Exemplary, the sensors are associated to the driving or driven pulley, or to the guide or to the slider.

Preferably, as shown by way of example in the figures, the frame 3 further comprises actuating means 60 acting upon a respective dispensing element 5a of a spray container 5, or upon respective dispensing elements 5a of the plurality 4 of spray containers, for automatically delivering the tanning substance from each spray container.

Preferably, these actuating means 60 comprise at least one control element 61 shifting between at least a first position in which it does not interact with a respective dispensing element 5a and a second position in which it pushes this dispensing element for automatic delivery.

Preferably, the actuating means comprise an actuator 65 and a control element 61 for each spray container, preferably actuated independently by the other control elements. Preferably the aforesaid control element is an eccentric cam 66 having a central portion 67 associated to the actuator and an operating surface 68, the actuator being configured for determining the passage of the eccentric cam between the aforesaid first position, in which the operating surface does not press on the respective dispensing element 5a of the spray container, and the aforesaid second position in which said operating surface presses the dispensing element.

Preferably, in the first position the operating surface is in contact with the dispensing element but does not exercise on it a push able to change its position. Thus the eccentric cam is always in contact with the respective dispensing element, both during the delivery of the substance contained in the container and during non-delivery, avoiding impacts between the eccentric cam and the dispensing element during the transition between the first and second position.

Preferably, the actuator 65 comprises a rotary motor (e.g. a DC motor or a stepper motor) provided with a drive shaft on which is fixed said central portion of the eccentric cam. Preferably the rotary motor comprises a set of gears (not shown) configured for generating the correct rotation of the drive shaft so as to determine the transition between said first and second position of the control element.

In the embodiment exemplary shown in FIG. 2, the control element 61 is basically shaped as a bar 62 which can simultaneously actuate the spray containers of one row 26 or 27 housed on the framework 20. When the bar is in the aforesaid second position, it acts by pushing the dispensing element 5a of each spray container of the row, thus causing the automatic delivery of the tanning substance contained in each spray container.

Preferably, the switching of the control element 61 between the first and second position is caused by a respective actuator (not shown) placed on the framework, made up for instance of a rotary or linear motor or of a hydraulic or pneumatic piston.

Preferably, the actuating means comprise more than one control element, e.g. two bars 62 as shown in FIGS. 2 and 3, wherein each control element acts upon a respective spray container or group of spray containers. In the example shown in the figure, the two bars 62 act each upon a respective row 26 and 27 of spray containers, and can be independently actuated so as to deliver the substance from the spray containers of the respective rows.

Alternatively, a single actuator can control in a coordinated mariner both bars causing the delivery of tanning substance from one or the other row of spray containers. With reference to the aforesaid example wherein the two opposed rows 26 and 27 generate a light and intense tanning, respectively, the actuation of one or the other row occurs in a mutually exclusive manner depending on the desired degree of artificial tanning. Alternatively, upon exhaustion of the tanning substance in one or more of the spray containers in a row, the switching of a bar from the second to the first position (with subsequent interruption of delivery) can coincide with the switching of the other bar from the first to the second position, thus starting the delivery of tanning substance from the spray devices of the respective row. By way of example, the two bars 62 in FIG. 2 can be connected, e.g. integrally, one with the other so as to obtain a coordinated movement thereof. Alternatively, in an embodiment of the framework that is not shown, the actuating means 60 comprise a slide having the aforesaid seat or plurality of seats with which the aforesaid spray container or plurality of spray containers are associated, which can shift between at least a first position in which the dispensing element or elements of the container or containers housed on the slide do not interact with a counter-element, and a second position in which the dispensing element or elements are pushed against this counter-element.

In the embodiment shown by way of example in FIGS. 1, 2 and 3, the seats on the frame are arranged so as to cause a basically horizontal arrangement of the spray containers, each with their own axis of longitudinal development basically parallel to the direction of longitudinal development 12a of the support 12. Preferably, the dispensing element of each spray container is shaped so as to direct delivery perpendicularly to the respective axis of longitudinal development. It is thus possible, by suitably arranging each container in its seat, to obtain a delivery of the tanning substance directed from top to bottom and towards the support. Alternatively, the seats can be arranged on the frame so as to cause an inclination of the spray containers contained therein, e.g. downwards (with the dispensing element in lower position, or preferably upper, with respect to the bottom of the spray container). This enables for instance to keep during use the remaining mixture in the containers within the container portion that is closer to the dispensing element, and to deliver the whole amount of mixture contained in the spray container.

In the embodiment shown by way of example in FIGS. 2 and 3, a bottom surface 23a of the trolley has on each seat 7 a respective hole 25 for letting through the mixture delivered from a respective spray container towards the support 12. It is thus possible to arrange the seats on the bottom surface 23a and to house the spray containers resting thereon, each with its own dispenser directed downwards.

Preferably the tanning device further comprises a heat source 70 configured for heating the skin of the user. Preferably, the device comprises one heat source for each spray container. Preferably, each heat source comprises a hot air generator 71, a duct 73 and an electric fan 72 (centrifugal or axial) for creating a stream of hot air exiting by the duct.

Preferably the heat source is configured for generating a stream of hot air directed in the same direction of delivery of the tanning substance from the spray containers and/or so that, in the translational movement of the framework with respect to the support, the stream of hot air reaches the skin of the user after the tanning substance delivered by the spray containers.

Preferably the hot air generator and the electric fan coincide in a sole device apt to generate and move hot air.

Preferably, in the embodiment exemplary shown in FIGS. 4, 5, 6 and 7, each seat 7 comprises at least one jaw 7a countershaped to the respective spray container and having a compliance that permit an elastic deformation of the jaw which allows to manually enter, by pressing, a spray container in the jaw and to manually remove, by pulling, the spray container by the jaw while maintaining, without pressure or pull, the container firmly housed in the jaw. Preferably the jaw has a shape of portion of a ring (exemplarily has a “C” conformation), preferably is made of plastic.

Preferably each seat comprises a pair of jaws 7a placed in such a way to wrap and hold the spray container respectively in correspondence of the bottom and the top of the container.

Preferably each container 5 of the device 1 is singularly associated with a respective delivery group 80 mounted to the framework 20 and comprising the respective seat 7, the respective actuating means 60 and the respective heat source 70 singularly dedicated to the respective container.

Preferably the seat and/or the actuating means are mounted to a respective box 81 fixed to the framework. Thus it is not necessary to assemble the delivery groups directly on the framework, facilitating subsequent maintenance or replacement of components associated with said box.

Preferably, each heat source is positioned in the respective delivery group, near the respective spray container and so as to generate a stream of hot air directed in the same direction of delivery of the spray container associated with it. Preferably, the framework has a curved shape (preferably a part of a circumference) and the delivery groups are distributed circumferentially on it, preferably angularly equally spaced between them. In this way, the framework evenly surrounds the user placed on the support and each dispensing element is substantially at the same distance by the user. In this way the substance delivered by the various spray containers cover the same distance, ensuring a uniform coverage of the skin of the user. Exemplary, the framework comprises five delivery groups.

In a possible embodiment, shown exemplary in FIG. 7, the aforesaid support 12 consists of a back 18 of a chair 112 structured for allowing the user to be in a lying position for receiving the tanning substance supplied by said plurality of spray containers or nozzles. Preferably, the chair comprises a seat 17 and the back 18 having a respective direction of longitudinal development 12a.

Preferably, in such embodiment, the trolley consists of a portal structure 29 comprising the aforesaid framework, a support member 33 and two uprights 31 and 32 in support of said framework 20. Preferably the uprights are arranged on opposite sides with respect to the support.

Preferably the support member lies behind the back. Preferably the support member is provided with two end portions 35 and 36 opposed each other and stiffly fixed each to a respective lower end of the respective upright. Preferably the portal structure develops transversally (preferably perpendicularly) to the direction of longitudinal development 12a. Preferably the framework surrounds the head and the trunk of a user lying on said chair and moving with respect to the chair along the direction of longitudinal development (12a).

Preferably, the framework 20 has the same technical features described for the embodiment of FIG. 4, but it is associated with the back and moves with respect to it.

Preferably, the aforesaid movement means 40 are positioned in the back and structured for moving the two uprights with respect to the back and for determining the movement of the portal structure from top to bottom and vice versa. Preferably, the movement means comprise a motor 41 and two guides 46, shown schematically in FIG. 7. Exemplary, the framework comprises three delivery groups 80. Preferably, the framework and/or the portal structure is manufactured by assembling plates opportunely shaped (obtained for example by bending or cutting) and joined by welding, screws or rivets. Preferably, these plates are provided with openings in correspondence of the seats, for the insertion and removal of spray containers, and of the heat sources, for the outflow of the air streams. Preferably, the plates are provided with air vents 39 for cooling the components inside the framework (for example, operation means, hot air generators, fans).

Preferably the device comprises a control panel 90, associated to the framework or to an upright or to the support or to the base, configured for allowing the control of the device by the user, for example for selecting the speed and/or the excursion of the translation of the framework and/or for setting the quantity of tanning substance that it is intended to be delivered toward the user. Preferably the control panel comprises a display and/or a keyboard.

Preferably, the device comprises a device for acoustic diffusion 91, for example a speaker, associated to the framework or to an upright or to the support or to the base, apt to diffuse music and/or acoustic signals and/or information about the state of the room of the tanning device.

Preferably, the tanning device can further comprise an electronic central unit (not shown) apt to manage the operation of the device, to interface with and monitor the aforesaid movement means 24 and 40, actuating means 60 and/or sensors and/or actuators 65, and to process delivery sequences for the tanning substance.

The operation of the device 1 includes placing at least one spray container 5 in the aforesaid seat 7 or a plurality of spray containers in the aforesaid plurality of seats. The user lies down on the support 12 and the device delivers the tanning substance towards at least one area of the user's body from a respective dispensing element of the spray container or of the plurality of spray containers. Upon exhaustion of the mixture in one or more spray containers, or upon obstruction or failure of a dispensing element, one or more spray containers are removed from the frame and one or more new spray containers containing tanning substance to be supplied, typically wholly similar to those removed, are associated with the frame.

In the embodiment shown by way of example in FIG. 1, the user switches, after a given time from the beginning of the treatment, from a lying supine position to a lying prone position, or vice versa, so that the tanning substance can reach and cover the whole body area of the user. In this case the tanning substance is delivered downwards.

In the embodiments shown by way of example above, delivery occurs automatically by means of the aforesaid actuating means acting upon the respective dispensing elements of each spray container.

1. A tanning device (1) comprising a resting base (2) for a user and a supporting frame (3) associated with said base (2) and configured for housing a plurality of delivery nozzles of a spray delivery system of a tanning substance, where said base (2) comprises a support (12) with substantially flat shape for enabling the user to lie down for receiving the tanning substance delivered from said delivery nozzles.

2. The device (1) according to claim 1, wherein said spray delivery system comprises a tank containing said tanning substance and a source of pressure for spraying said tanning substance from said delivery nozzles toward the user in a direction from the top toward the bottom, said frame comprising a framework overlying said support and provided with said plurality of nozzles, so that the tanning substance is delivered towards said support, the framework being able to translate with respect to said support in a direction parallel to a direction of longitudinal development of said support.

3. The device (1) according to claim 1, wherein the frame (3) comprises a plurality of seats (7) apt to removably house a plurality of spray containers (5) for said tanning substance and provided with said delivery nozzles for delivering said tanning substance automatically towards the user, said frame (3) comprising a framework (20) overlying said support (12) and provided with said plurality of seats (7), so that the tanning substance is delivered towards said support (12), the framework being able to translate with respect to said support in a direction parallel to a direction of longitudinal development (12a) of said support (12).

4. The device (1) according to claim 2, wherein said frame (3) comprises at least a sliding rail (22; 46) developing along said direction of longitudinal development (12a) of the support (12), a trolley (23; 29) provided with said plurality of seats (7) or with said nozzles and slidingly engaged with said rail (22; 46), and movement means (24; 40) for said trolley (23; 29) along said rail (22; 46).

5. The device (1) according to claim 4, wherein said support (12) has a substantially horizontal development and said trolley consists of a portal structure (29) comprising said framework (20), a support member (33) and two uprights (31, 32) in support of said framework (20) arranged on opposite sides with respect to the support, the support member lying below the support and being provided with two end portions (35) and (36) opposed each other and stiffly fixed each to a respective lower end of the respective upright, said portal structure developing transversally to the direction of longitudinal development (12a) and said portal structure extending to a height, above the support (12), apt to allow the user to position himself lying on such support.

6. The device (1) according to the preceding claim 5, where the frame comprises two guides (46), developing parallel to the longitudinal development in correspondence of the longitudinal opposed sides of the support, and the support member (33) comprises at its two end portions (35) and (36) two sliders (47) slidingly inserted into the respective guide, where said movement means (40) are structured for moving the support member (33) with respect to the base and for determining the translation of the portal structure (29) parallel to the direction of longitudinal development (12a).

7. The device (1) according to claim 4, wherein said support (12) consists of a back (18) of a chair (112) structured for allowing the user to be in a lying position for receiving the tanning substance supplied by said plurality of spray containers or nozzles, the trolley consisting of a portal structure (29) comprising said framework, a support member (33) and two uprights (31, 32) in support of said framework (20) arranged on opposite sides with respect to the support, the support member lying at the back of the back and being provided with two end portions (35) and (36) opposed each other and stiffly fixed each to a respective lower end of the respective upright, said portal structure developing transversally to the direction of longitudinal development (12a) and said framework surrounding the head and the trunk of a user lying on said chair and moving with respect to the chair along said direction of longitudinal development (12a).

8. The device (1) according to claim 3 any one of the claims from 3 to 7, wherein said frame (3) further comprises actuating means (60) configured for acting upon a respective dispensing element (5a) of each spray container (5) or of said plurality of spray containers (5), for automatically delivering the tanning substance from said spray containers (5), said actuating means (60) comprising at least one control element (61) shifting between at least a first position in which it is not apt to interact with said corresponding dispensing element (5a), and a second position in which it is apt to push said dispensing element (5a) for obtaining said automatic delivery.

9. The device (1) according to claim 8, wherein the actuating means comprise an actuator (65) and a control element (61) for each spray container, said control element being an eccentric cam (66) having a central portion (67) associated to the respective actuator and an operating surface (68), the actuator being configured for determining the passage of the eccentric cam between said first position, in which the operating surface does not press on the respective dispensing element (5a) of the spray container, and said second position in which said operating surface presses the dispensing element.

10. The device (1) according to claim 1, wherein the framework comprises a heat source (70) configured for heating the skin of the user, the heat source being a generator of a stream of hot air, having a temperature preferably greater than 30° C., or a source of an infrared radiation.

11. The device (1) according to claim 10, wherein said heat source (70) comprises a hot air generator (71), a duct (73) and an electric fan (72) for creating a stream of hot air exiting by the duct, the heat source being configured for generating a stream of hot air directed in the same direction of delivery of the tanning substance from the spray containers and so that, in the translational movement of the framework with respect to the support, the stream of hot air reaches the skin of the user after the tanning substance delivered by the spray containers.

12. The device (1) according to claim 3, wherein each container (5) is singularly associated with a respective delivery group (80) mounted to the framework (20) and comprising the respective seat (7), the respective actuating means (60) and/or the respective heat source (70) singularly dedicated to the respective container.

13. The device (1) according to claim 3, comprising said plurality of spray containers (5), each spray container containing a pressurized mixture containing at least said tanning substance, typically dihydroxyacetone (DHA), and being equipped with a dispensing element (5a) for atomizing said mixture.

14. A tanning method comprising the following steps:
arranging a tanning device (1) according to claim 3;
placing said plurality of spray containers (5) in said plurality of seats (7);
deliver the tanning substance, towards at least one area of the user's body, from a respective dispensing element (5a) of said plurality of spray containers (5) by actuating the respective dispensing elements (5a);
removing one or more spray containers (5) of said plurality of spray containers (5) from said frame (3) and, as replacement, associating with said frame (3) one or several spray containers (5) containing tanning substance to be delivered.
